(19) United States
(12) Patent Application Publication
(10) Pub. No.: US 2014/0012127 A1
(43) Pub. Date: Jan. 9, 2014

(54) HEARING PROTECTION APPARATUS AS WELL AS A MEDICAL IMAGING APPARATUS HAVING THE HEARING PROTECTION APPARATUS AND A METHOD FOR DETECTING MOVEMENT OF A PATIENT'S HEAD

(71) Applicants: Stephan Biber, Erlangen (DE); Lars Lauer, Neunkirchen (DE)

(72) Inventors: Stephan Biber, Erlangen (DE); Lars Lauer, Neunkirchen (DE)

(21) Appl. No.: 13/933,386

(22) Filed: Jul. 2, 2013

(30) Foreign Application Priority Data
Jul. 4, 2012 (DE) 102012211621.4

(51) Int. Cl. A61B 5/00; A61B 5/055; A61F 11/14; A61B 5/11; A61F 11/08

(52) U.S. Cl. CPC A61B 5/6817; A61B 5/11; A61F 11/08; A61F 11/14; A61B 5/0004; A61B 5/0555; A61B 5/7285
USPC 600/411; 600/595

(57) ABSTRACT

A hearing protection apparatus includes a first ear coupling unit, which is designed for positioning on a first ear of a user, a second ear coupling unit, which is designed for positioning on a second ear of the user, and a sound protection for at least partial shielding of sound waves, wherein the hearing protection apparatus has at least one motion sensor unit.

### CROSS REFERENCE TO RELATED APPLICATIONS

The present application claims priority to German Pattent Office application No. 10 2012 211621.4 filed Jul. 4, 2012, the entire content of which is hereby incorporated herein by reference.

### FIELD OF INVENTION

The present invention relates to a hearing protection apparatus having a first ear coupling unit, which is designed for positioning on a first ear of a user, a second ear coupling unit, which is designed for positioning on a second ear of the user, and a sound protection unit for at least partial shielding of sound waves.

### BACKGROUND OF INVENTION

It is important for magnetic resonance imaging that a patient does not execute any movement for the entire duration of magnetic resonance measuring. Patient movement during magnetic resonance measuring can produce artifacts in the magnetic resonance images, which can then result in misinterpretation and/or reduced conclusiveness when performing a medical evaluation of the magnetic resonance images. It may also be that magnetic resonance measuring has to be repeated due to unwanted patient movement. It is often problematic, in particular during magnetic resonance measuring on patients with a tendency to claustrophobia and/or pain patients and/or children, for the patient to remain lying still for the duration of magnetic resonance measuring.

Methods are already known, with which movement of the patient's body is detected by means of magnetic resonance measuring and a change is then made to the course of a measuring sequence, for example an adjustment of the gradient plane.

However such methods or such a correction have to be developed individually for every magnetic resonance sequence.

It is also known to detect patient movement by means of a sensor unit. Thus for example in Brian Andrews-Shigaki et al.: “Characterization of Head Motion in the MR Environment”, published at a method is disclosed in which additional marker elements are positioned on the patient to detect patient movement. However this can result in more work for operators.

A method is also known from Oline Olesen et al.: “Motion tracking for medical imaging: a nonvisible structured light approach”, IEEE Trans. On Med. Imaging, January 2012, in which patient movement is determined by means of optical strip projection. However this method can only be implemented with difficulty due to a lack of space in a head coil for example. The method is also only insufficiently accurate when detecting movement.

It is also particularly problematic to detect the patient's head movement for the duration of the magnetic resonance examination of the head region of the patient in particular. The patient's head is preferably supported in a head coil during the magnetic resonance examination. This restricts the freedom of movement of the patient's head but the lack of space within the head coil also means that it is very difficult to detect movement of the patient's head during the magnetic resonance examination.

### SUMMARY OF INVENTION

The object of the present invention is in particular to provide an apparatus, which allows simple and space-saving detection of a patient's head movement. The object is achieved by the features of the independent claims. Advantageous embodiments are described in the dependent.

The invention is based on a hearing protection apparatus having a first ear coupling unit, which is designed for positioning on a first ear of a user, a second ear coupling unit, which is designed for positioning on a second ear of the user, and a sound protection unit for at least partial shielding of sound waves.

It is proposed that the hearing protection apparatus has at least one motion sensor unit, allowing movement of the patient's head to be detected particularly quickly and directly during a medical imaging examination of the head and/or a head region of the patient, in particular during a magnetic resonance examination of the patient's head. This also allows correction measures to be taken particularly quickly due to the movement of the patient's head, for example asking the patient to lie still and/or taking movement corrections into account when evaluating the data and/or restarting the medical imaging examination due to patient movement. It is also advantageously possible to dispense with additional markers to be positioned on the user, in particular on the head of the patient, and/or additional units for detecting head movement, thereby also achieving a simple examination sequence by using the hearing protection apparatus for detecting movement of the patient's head. Integration of the motion sensor unit within the hearing protection unit allows a particularly compact and space-saving arrangement of the motion sensor unit within a medical imaging apparatus in particular for medical imaging examinations, preferably magnetic resonance examinations. The two ear coupling units are preferably designed for direct positioning on the ears of the user, in particular the patient, with a hearing protection function also only being active when the ear coupling units are positioned directly on and/or brought into contact with the ears of the patient.

It is further proposed that the motion sensor unit has at least one sensor element for detecting a translational movement and/or a rotational movement of the user's head. This allows any type of movement of the head of the user, in particular the patient, to be detected particularly quickly during a medical imaging examination of the patient's head. The movement of the user's head is preferably detected along three translational directions and along three rotational directions. To this end the motion sensor unit particularly advantageously has at least one acceleration sensor element and/or at least one gyroscopic sensor element. The at least one sensor element can also be formed by a sensor element, which emits a sensor signal that is received by a further sensor element, which is disposed for example on a magnetic resonance device, and/or comprises a sensor element, which comprises a reflector element and/or a marker element, etc.

Particularly advantageous, in particular complete, detection of the movement of the patient's head can be achieved, if the motion sensor unit has at least two sensor elements. A first of the at least two sensor elements here is preferably designed to detect the translational movement, in particular along three different spatial directions, and a second of the at least two sensor elements is designed to detect the rotational movement, in particular along three rotational directions. This also allows a redundant motion sensor unit to be provided for detecting the movement of the head of the user, in particular the patient.

The at least two sensor elements here can be disposed within the first ear coupling unit or within the second ear coupling unit, thereby providing a particularly compact electronic system for further signal processing and/or an electronic data transmission system, which is preferably disposed within the first and second ear coupling unit, for the motion sensor unit.

However at least a first of the at least two sensor elements is particularly advantageously disposed within the first ear coupling unit and at least a second of the at least two sensor elements is disposed within the second ear coupling unit. This allows a particularly high level of accuracy to be achieved when detecting movement of the head of the user, in particular the patient, as the movement of the head of the user, in particular the patient, can be detected or sensed at the same time at different locations. For example an acceleration sensor element can be disposed within the first ear coupling unit and a sensor element for detecting a rotational movement can be disposed within the second ear coupling unit. It is also conceivable for an acceleration sensor element or a sensor element for detecting the rotational movement, etc. to be disposed respectively within the first ear coupling unit and also within the second ear coupling unit.

In a further embodiment of the invention it is proposed that the motion sensor unit has at least one electronic control unit, thereby allowing a particularly compact hearing protection apparatus for detecting movement of the head of the user and/or the patient to be achieved. The electronic control unit here is particularly advantageously disposed within the first ear coupling unit and/or the second ear coupling unit, in particular within the ear coupling unit having the at least one sensor element. The electronic control unit can comprise an evaluation unit and/or a data filter unit and/or an amplifier unit and/or further electronic control elements that appear expedient to the person skilled in the art.

A particularly fast and direct data transmission to for example a control unit and/or an evaluation unit of a medical imaging apparatus can advantageously be achieved if the motion sensor unit has at least one data transmission unit. The data transmission unit is preferably disposed directly within the ear coupling unit having the sensor element. To this end the data transmission unit particularly advantageously has at least one antenna element, allowing wireless and/or cableless data transmission in particular to a control unit and/or an evaluation unit of a medical imaging apparatus to be achieved, thereby advantageously allowing additional cables for data transmission to be dispensed with. This also simplifies patient preparation for the medical imaging examination, for example a magnetic resonance examination, for clinical operators.

It is further proposed that the motion sensor unit has at least one power storage unit to supply power to the sensor elements, allowing the motion sensor unit in particular to be operated in a cableless and/or wireless manner The power storage unit is preferably disposed directly within the ear coupling unit having the sensor element. The power storage unit here can comprise conventional, in particular rechargeable, batteries.

The number of additional units to be positioned on the patient for the medical imaging examination can advantageously be reduced, if the hearing protection apparatus has a communication unit for transmitting communication signals to a user, in particular a patient. This allows instructions, for example, to be transmitted to the patient by means of the communication unit during the medical imaging examination. It is also conceivable for the communication unit to be designed to transmit music signals, in particular to calm the patient during the medical imaging examination. In this process the data transmission unit of the communication unit transmits data from a control unit and/or voice input unit, for example for clinical operators to input voice signals, of the medical imaging apparatus to the hearing protection apparatus, with the hearing protection apparatus here also having the function of a headphone apparatus.

A particularly compact hearing protection apparatus can be achieved, if the communication unit has a data transmission unit, which is configured at least to some degree as a single piece with the data transmission unit of the motion sensor unit.

In a further embodiment of the invention it is proposed that the first ear coupling unit and/or the second ear coupling unit comprise(s) an earplug, allowing the hearing protection unit to be configured in a particularly compact manner The earplugs preferably have a shape similar to headphones that can be inserted into the auditory canal. Such a hearing protection apparatus allows movement of the patient's head to be detected particularly effectively, as the arrangement of the first ear coupling unit and/or the second ear coupling unit in an auditory canal of the patient allows the motion sensor unit, in particular the individual sensor elements, to register and/or detect any type of head movement. Alternatively the first ear coupling unit and/or the second ear coupling unit can also comprise an earmuff. The earmuff is preferably disposed around the ear of the operator, in particular the patient, for effective hearing protection.

The invention is also based on a magnetic resonance apparatus having a hearing protection apparatus as claimed in one of claims 1 to 15. This allows movement of the patient's head to be detected particularly quickly and directly during a magnetic resonance examination of a head region of the patient. It also allows correction measures to be taken particularly quickly due to the movement of the patient's head, for example asking the patient to lie still and/or taking movement corrections into account when evaluating the data and/or restarting medical imaging measuring due to patient movement. It is also advantageously possible to dispense with additional markers to be positioned on the user, in particular on the head of the patient, and/or additional units for detecting head movement, thereby also achieving a simple examination sequence by using the hearing protection apparatus for detecting movement of the patient's head.

It is further proposed that the magnetic resonance apparatus has a data transmission unit, which is designed for data transmission with the motion sensor unit of the hearing protection apparatus. The data transmission unit preferably comprises at least one antenna element for cableless and/or wireless data transmission between the motion sensor unit and the data transmission unit, so that additional data transmission cables can advantageously be dispensed with. The data transmission unit preferably transmits the data from the motion sensor unit of the hearing protection apparatus to an evaluation unit and/or a control unit and/or a further motion sensor unit, etc.

In one advantageous development of the invention it is proposed that the magnetic resonance apparatus has at least one sensor unit, which detects a sensor signal, which is emitted and/or reflected by at least one sensor element of the hearing protection apparatus. This allows the hearing protection apparatus to be kept particularly compact, in that at least one subregion of a motion sensor unit for detecting movement of the patient's head can be disposed outside the hearing protection apparatus. It is particularly advantageous here for the at least one sensor element to be disposed on a housing of the magnetic resonance apparatus that encloses a patient accommodation region, so that there is the shortest transmission distance possible between the hearing protection apparatus and the sensor element.

It is further proposed that the magnetic resonance apparatus has a data evaluation unit, which is designed to evaluate the sensor signals. The data evaluation can take place directly in the magnetic resonance apparatus here, thereby allowing the hearing protection apparatus to be kept particularly compact. It also allows a control unit of the magnetic resonance apparatus to perform a direct analysis of the evaluated data, for example whether movement of the patient's head takes place during the magnetic resonance examination and if there is head movement, what type of head movement it is.

If the magnetic resonance apparatus has a control unit, which is designed to control magnetic resonance measuring and takes into account the evaluated data of a data evaluation unit when controlling magnetic resonance measuring, it is particularly advantageously possible to respond directly to changes in patient position, in particular the position of the patient's head. Also after identifying movement of the patient's head and/or a change in the position of the patient's head the control unit can institute the necessary correction steps. For example the patient can be asked, by the outputting of optical and/or acoustic instructions, to lie still on a patient support apparatus. Magnetic resonance measuring can also be terminated immediately and/or restarted and/or just individual partial measurements can be repeated. This also allows the measuring time for the patient to be shortened, as there is no need to wait until the end of magnetic resonance measuring to decide whether the measurement in question can be used for medical analysis. This also allows patient throughput to be advantageously increased at the magnetic resonance device, thereby allowing particularly effective usage of the magnetic resonance apparatus.

The invention is also based on a method for detecting movement of a patient's head, in particular for magnetic resonance measuring, comprising the following method steps:
Detecting a sensor signal by means of a motion sensor unit, which is disposed at least partially within an ear coupling unit of a hearing protection apparatus,
Determining movement information by evaluating the sensor signal by means of a data evaluation unit,
Transmitting the movement information to a control unit and
Generating a control signal for magnetic resonance measuring as a function of the movement information.

This allows movement of the patient's head to be detected particularly quickly and directly during a magnetic resonance examination of the patient's head. Correction steps required due to the movement of the patient's head can also be taken particularly quickly, for example asking the patient to lie in particular perfectly still and/or with little movement and/or taking movement corrections into account when evaluating the data and/or restarting the magnetic resonance examination due to patient movement. It is also advantageously possible to dispense with additional markers to be positioned on the user, in particular on the head of the patient, and/or additional units for detecting head movement, thereby also achieving a simple examination sequence by using the hearing protection apparatus for detecting movement of the patient's head.

### BRIEF DESCRIPTION OF THE DRAWINGS

Further advantages, features and details of the invention will emerge from the exemplary embodiments described below and based on the drawings, in which:

FIG. 1 shows a schematic diagram of an arrangement of a first exemplary embodiment of an inventive hearing protection apparatus on a user,

FIG. 2 shows a schematic diagram of the inventive hearing protection apparatus from FIG. 1,

FIG. 3 shows a schematic diagram of an alternative embodiment of the hearing protection apparatus to FIG. 2,

FIG. 4 shows a schematic diagram of an arrangement of a third exemplary embodiment of an inventive hearing protection apparatus on a user,

FIG. 5 shows the schematic diagram of the inventive hearing protection apparatus from FIG. 4,

FIG. 6 shows a schematic diagram of an inventive magnetic resonance apparatus having a hearing protection apparatus,

FIG. 7 shows a schematic diagram of an alternative embodiment of the magnetic resonance apparatus having a fourth exemplary embodiment of an inventive hearing protection apparatus,

FIG. 8 shows the fourth exemplary embodiment of the inventive hearing protection apparatus and

FIG. 9 shows a schematic diagram of an inventive method.

### DETAILED DESCRIPTION OF INVENTION

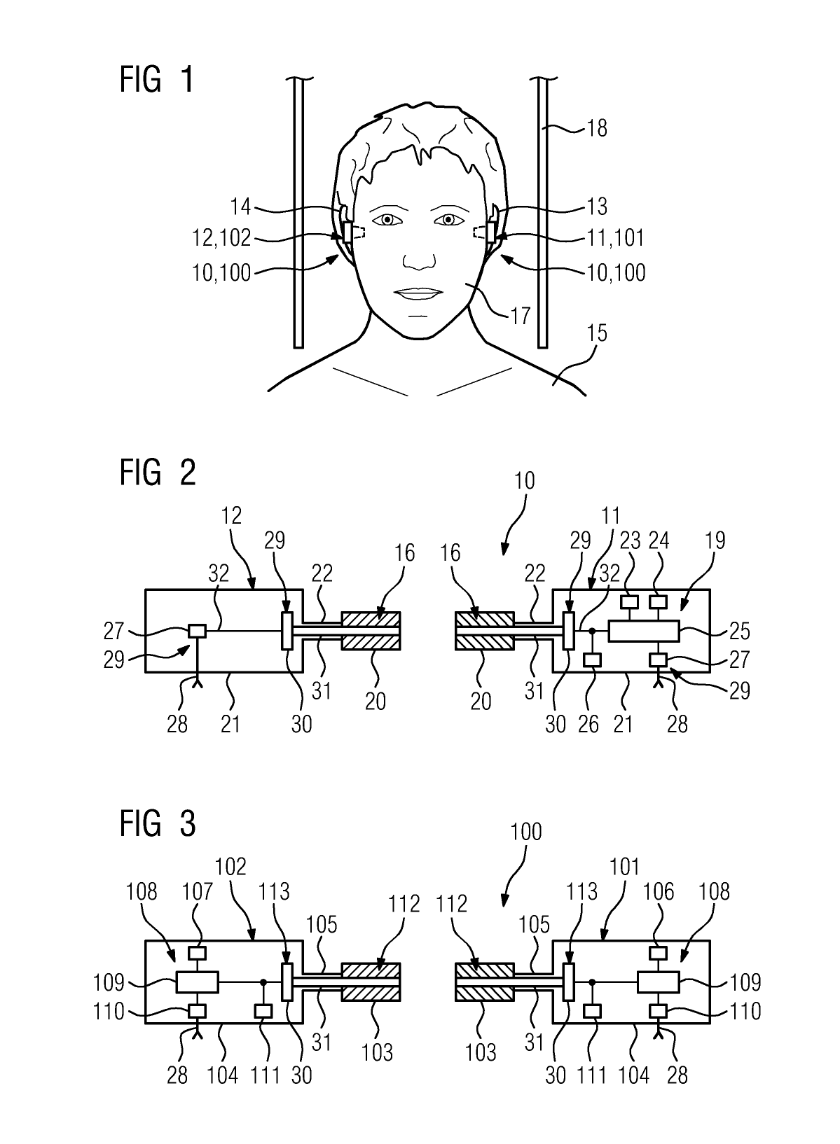

FIGS. 1 and 2 show schematic diagrams of a first exemplary embodiment of an inventive hearing protection apparatus 10. The hearing protection apparatus 10 comprises a first ear coupling unit 11 and a second ear coupling unit 12, the first ear coupling unit 11 being designed to position and/or arrange the hearing protection apparatus 10 on a first ear 13 of a user and the second ear coupling unit 12 being designed to position and/or arrange the hearing protection unit 10 on a second ear 14 of the user. The user is a patient 15 in the present instance. The first ear coupling unit 11 and the second ear coupling unit 12 here are formed by headphones that can be inserted into the auditory canal.

The hearing protection apparatus 10 further comprises a sound protection unit 16 for the at least partial shielding of sound waves, so that the sounds waves are shielded from the patient 15 after the ear coupling units 11, 12 have been positioned on the ears 13, 14 of the patient 15. The sound protection unit 16 is disposed within the first ear coupling unit 11 and also within the second ear coupling unit 12 (FIG. 2).

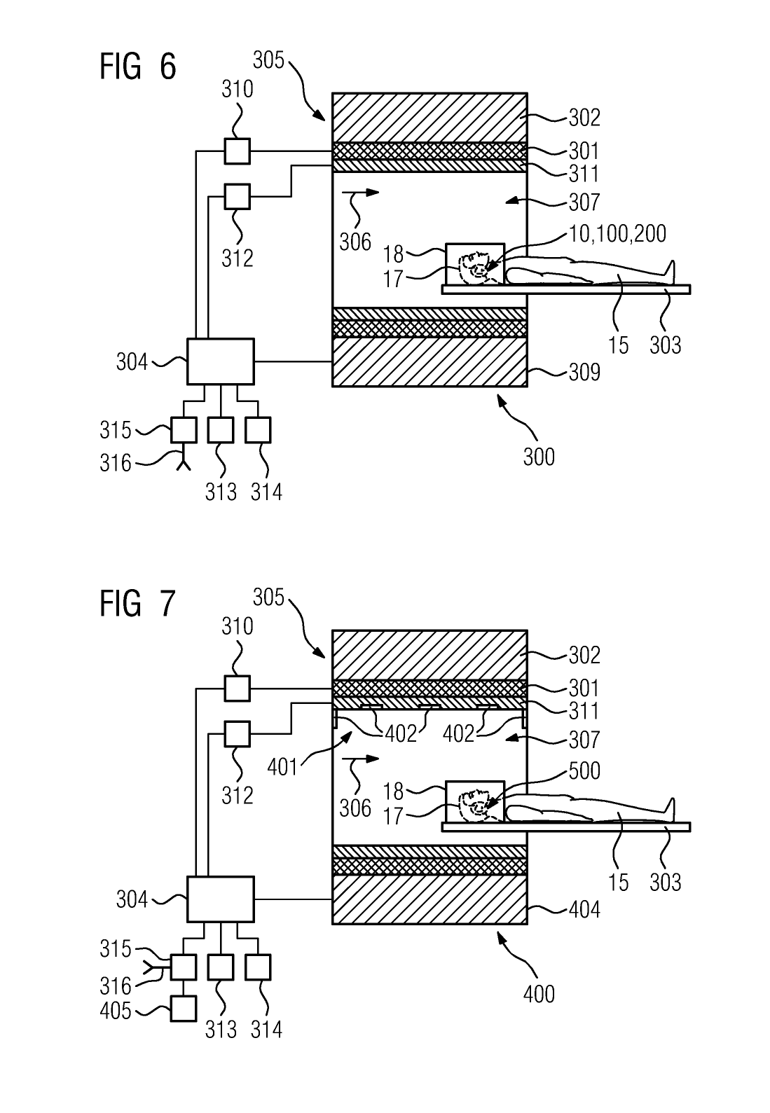

Such a hearing protection apparatus 10 is used in particular during magnetic resonance examinations, in which loud noises occur due to the interaction of a gradient coil unit 301 of the magnetic resonance apparatus 300 with a main magnet 302 of the magnetic resonance apparatus 300 (FIG. 6). If the magnetic resonance examination includes an examination of the head 17 of the patient 15, a local head coil 18 is also disposed around the head 17 of the patient 15 to transmit excitation signals and/or receive magnetic resonance signals. It is important for a successful magnetic resonance examination that the patient 15 lies as still as possible on a patient support apparatus 303 for the duration of the magnetic resonance examination. If the patient 15 moves during the magnetic resonance examination it can result in misinterpretations in the magnetic resonance images and/or repetition of the magnetic resonance examination.

The inventive hearing protection apparatus 10 therefore has a motion sensor unit 19, which can be used to detect movement of the head 17 of the patient 15, as shown in FIG. 2. The at least partial integration of the motion sensor unit 19 within the hearing protection apparatus 10 allows a particularly space-saving arrangement of the motion sensor unit 19 within the magnetic resonance apparatus 300 to be achieved, in particular for head examinations, in which a head coil unit 18 is also used.

The hearing protection apparatus 10 is shown in more detail in FIG. 2. The first ear coupling unit 11 and the second ear coupling unit 12 respectively comprise a first hearing protection unit 20, a second hearing protection unit 21 and a connecting unit 22, the connecting unit 22 connecting the first hearing protection unit 20 to the second hearing protection unit 21. To this end the connecting unit 22 can comprise a fixed, in particular rigid, connection between the first hearing protection unit 20 and the second hearing protection unit 21 and/or a flexible connection between the first hearing protection unit 20 and the second hearing protection unit 21, so that the first hearing protection unit 20 can be moved relative to the second hearing protection unit 21.

The first hearing protection unit 20 is disposed in a position on the patient 15 within the ear 13, 14 of the patient 15. The second hearing protection unit 21 is disposed in the position on the patient 15 outside the ear 13, 14 but resting on the ear 13, 14 of the patient 15.

The motion sensor unit 19 is disposed within the second hearing protection unit 21 of one of the two ear coupling units 11 and comprises two sensor elements 23, 24. A first of the two sensor elements 23 is formed by a motion sensor element, which detects a translational movement of the head 17 of the patient 15 along three different spatial directions. A second of the two sensor elements 24 is formed by a gyroscopic sensor element, which detects a rotational movement of the head 17 of the patient 15 along three different spatial directions. Further configurations of the sensor elements 23, 24 that appear expedient to the person skilled in the art are also always conceivable.

The motion sensor unit 19 further comprises an electronic control unit 25, a power storage unit 26 and a data transmission unit 27, which are likewise disposed within the second hearing protection unit 21 of the ear coupling unit 11. The data transmission unit 27 has an antenna element 28, for wireless and/or cableless data transmission of the sensed sensor data for example to a control unit 304 and/or an evaluation unit of the magnetic resonance apparatus 300 (FIG. 6). In the present exemplary embodiment the power storage unit 26 comprises a conventional, in particular rechargeable, battery.

The electronic control unit 25 comprises a data evaluation unit, a data filter unit and/or an amplifier unit (not shown in detail). The electronic control unit 25 can also comprise further electronic components and/or units that appear expedient to the person skilled in the art.

The hearing protection apparatus 10 also has a communication unit 29, which allows communication between an operator and the patient 15 at least to some degree.

The communication unit 29 comprises two data transmission units 27, which are disposed respectively within the second hearing protection units 21 of the two ear coupling units 11, 12. The two data transmission units 27 each have an antenna element 28. A transmission of communication data also thus takes place by means of the wireless and/or cableless antenna element 28. The communication unit 29 can be used to transmit instructions from the operator to the patient 15. It is also possible for the communication unit 29 to transmit calming music to the patient 15 for the duration of the medical imaging examination in order to calm said patient 15. In the present exemplary embodiment the data transmission unit 27 disposed within the ear coupling unit 11 is configured as a single piece with the data transmission unit 27 of the motion sensor unit 19.

The communication unit 29 also comprises two sound transducer units 30 and two sound tube elements 31. The sound tube elements 31 extend respectively from the sound transducer units 30, which are disposed within the second hearing protection units 21 of the ear coupling units 11, 12, to an end region of the first hearing protection units 20 facing away from the second hearing protection units 21. Disposed within the first hearing protection units 20 are sound-damping units of the sound protection units 16, which shield the sound tube elements 31 radially outward against sound waves.

The individual units and/or elements, which are disposed within the second hearing protection units 21, are connected to one another by way of an internal signal transmission unit 32.

The arrangement of the motion sensor unit 19 within the hearing protection apparatus 10 means that it is possible to detect movement of the head 17 of the patient 15 directly in particular during a magnetic resonance examination, as because of its arrangement on the head 17 of the patient 15 the hearing protection apparatus 10 moves with the head 17 of the patient 15. The sensor elements 23, 24 hereby detect movement of the head 17 of the patient 15 directly. The electronic control unit 25 is used to at least partially evaluate and further process the detected data, which is then transmitted from the data transmission unit 27 to the control unit 304 of the magnetic resonance apparatus 300. The power storage unit 26 supplies the sensor elements 23, 24, the data transmission unit 27, the electronic control unit 25 and the communication unit 29 with electrical energy during operation of the hearing protection apparatus 10.

FIG. 3 shows an alternative embodiment of the hearing protection apparatus 100 to FIG. 2. The description which follows is essentially limited to the differences from the exemplary embodiment in FIG. 2, with reference being made to the description of the exemplary embodiment in FIG. 2 in respect of identical components, features and functions. Essentially identical components, features and functions are in principle shown with identical reference characters.

The hearing protection apparatus 100 likewise has a first ear coupling unit 101 and a second ear coupling unit 102, the two ear coupling units 101, 102 respectively having a first hearing protection unit 103, a second hearing protection unit 104 and a connecting unit 105. The second hearing protection units 104 each have a sensor element 106, 107 of the motion sensor unit 108. Each of the two second hearing protection units 104 also has an electronic control unit 109, a data transmission unit 110 and a power storage unit 111 in addition to the sound protection unit 112 and the communication unit 113. The further embodiment of the ear coupling units 101, 102 corresponds essentially to the embodiment of the ear coupling units 11, 12 of the hearing protection unit 10 from FIG. 2.

The sensor element 106 disposed within the first ear coupling unit 101 is formed by a motion sensor element and is designed to detect a translational movement along the three spatial directions. The sensor element 107 disposed within the second ear coupling unit 102 is formed by a gyroscopic sensor element and is designed to detect a rotational movement along the three spatial directions.

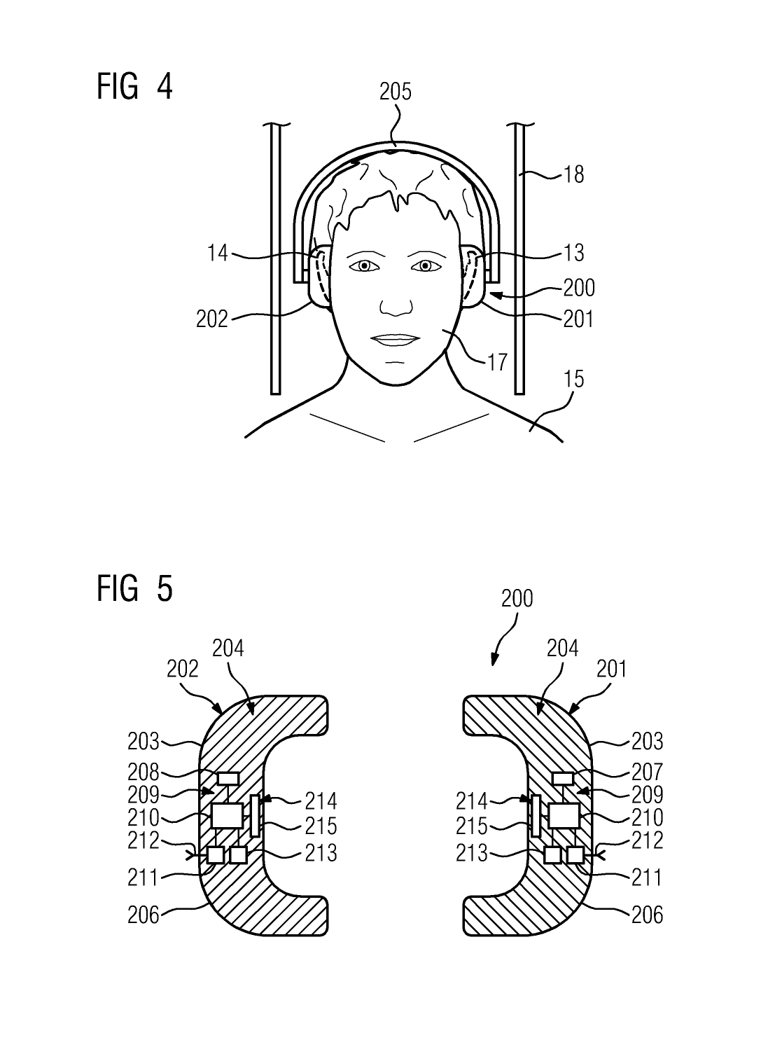

FIGS. 4 and 5 show an alternative exemplary embodiment of the hearing protection apparatus 200 for a magnetic resonance examination to FIGS. 1 to 3. The description which follows is essentially limited to the differences from the exemplary embodiments in FIGS. 1 to 3, with reference being made to the description of the exemplary embodiment in FIGS. 1 to 3 in respect of identical components, features and functions. Essentially identical components, features and functions are in principle shown with identical reference characters.

The hearing protection apparatus 200 in FIGS. 4 and 5 differs in the embodiment and shape of a first ear coupling unit 201 and in the embodiment and shape of a second ear coupling unit 202 from the hearing protection apparatuses 10, 100 in FIGS. 1 to 3. The first ear coupling unit 201 and also the second ear coupling unit 202 respectively comprise an earmuff 203. The earmuffs 203 comprise the sound protection unit 204, which in the present exemplary embodiment comprises a sound-damping material. The two earmuffs 203 are also connected to one another by means of yoke 205.

Disposed within each of the earmuffs 203 is a sensor element 207, 208 of the motion sensor unit 209, enclosed by a housing 206 of the earmuffs 203. Disposed in a first earmuff 203 is a sensor element 207 formed by a motion sensor element and disposed in a second earmuff 203 is a sensor element 208 formed by a gyroscopic sensor element. The two sensor elements 207, 208 allow the detection of a translational movement of the head 17 of the patient 15 along three spatial directions and the detection of a rotational movement along three rotational directions during operation of the hearing protection apparatus 200.

The motion sensor unit 209 further comprises two electronic control units 210, two data transmission units 211, which each have at least one antenna element 212 for wireless and/or cableless data transmission, and two power storage units 213, with one of the electronic control units 210, one of the data transmission units 211 and one of the power storage units 213 respectively being disposed in one of the two earmuffs 203 respectively.

The hearing protection apparatus 200 also has two communication units 214, with one of the two communication units 214 respectively being disposed in one of the earmuffs 203. The communication units 214 each comprise a sound transducer unit 215 and a membrane unit (not shown in detail) for outputting acoustic signals. The mode of operation and the arrangement of the individual structural units and/or structural elements to one another within the hearing protection unit 200 are configured in the same way as described in relation to FIGS. 2 and 3.

Provision can also be made for one of the two earmuffs 203 to enclose the motion sensor unit 209 completely, as described in relation to FIG. 2.

FIG. 6 shows a schematic diagram of the inventive magnetic resonance apparatus 300 having the hearing protection apparatus 10, 100, 200. The hearing protection apparatus 10, 100, 200 is configured according to one of the embodiments from FIG. 1 to FIG. 5.

The magnetic resonance apparatus 300 comprises a magnet unit 305 having the main magnet 302 for generating a powerful and in particular constant main magnetic field 306. The magnetic resonance apparatus 300 also has a cylindrical patient accommodation region 307 to accommodate the patient 15, the patient accommodation region 307 being enclosed in a peripheral direction by the magnet unit 305. The patient 15 can be moved into the patient accommodation region 307 by means of the patient support apparatus 303 of the magnetic resonance apparatus 300. To this end the patient support apparatus 303 is disposed in a movable manner within the magnetic resonance apparatus 300. The magnetic resonance apparatus 300 also has a housing unit 309 enclosing the magnet unit 305.

The magnet unit 305 also has the gradient coil unit 301 for generating magnetic field gradients, which is used for spatial encoding during imaging. The gradient coil unit 301 is controlled by means of a gradient control unit 310. The magnet unit 305 also has a high-frequency antenna unit 311 and a high-frequency antenna control unit 312 to excite a polarization, which is established in the main magnetic field 306 generated by the main magnet 302. The high-frequency antenna unit 311 is controlled by the high-frequency antenna control unit 312 and radiates high-frequency magnetic resonance sequences into an examination space, which is formed essentially by the patient accommodation region 307. This deflects the magnetization from its equilibrium position.

To control the main magnet 302, the gradient control unit 310 and to control the high-frequency antenna control unit 312, the magnetic resonance apparatus has the control unit 304 formed by a computation unit. The control unit 304 controls the magnetic resonance apparatus 300 centrally, for example the performance of a predetermined imaging gradient echo sequence. Control information such as imaging parameters for example, as well as reconstructed magnetic resonance images can be displayed to an operator on a display unit 313, for example on at least one monitor, of the magnetic resonance apparatus 300. The magnetic resonance apparatus 300 also has an input unit 314, which can be used by an operator to input information and/or parameters during a measuring operation.

The magnetic resonance apparatus 300 also has a data transmission unit 315 with an antenna element 316 for wireless and/or cableless data transmission with the data transmission unit 27, 110, 211 of the hearing protection apparatus 10, 100, 200. The data evaluated by the electronic control unit 25, 109, 210 is transmitted by way of the data transmission units 27, 220, 211, 315 to the control unit 304 of the magnetic resonance apparatus 300. The control unit 304 takes into account the evaluated data from the data evaluation unit of the electronic control system 25, 109, 210 when controlling magnetic resonance measuring. If movement of the head 17 of the patient 15 takes place during the magnetic resonance examination of the head region, this is first identified by the control unit 304 by means of the transmitted data from the hearing protection apparatus 10, 100, 200. The control unit 304 then automatically institutes correction steps in respect of the unwanted movement of the patient 15, in particular in respect of the unwanted head movement. These correction steps can include stopping measuring and/or restarting magnetic resonance measuring. The correction steps can also include a request to the patient 15 to remain as still as possible on the patient support apparatus 308 during magnetic resonance measuring.

It is also conceivable for the possible correction steps to be displayed by the control unit 304 to a clinical operator for selection by way of the output unit 313, so that the clinical operator can select one of the correction steps.

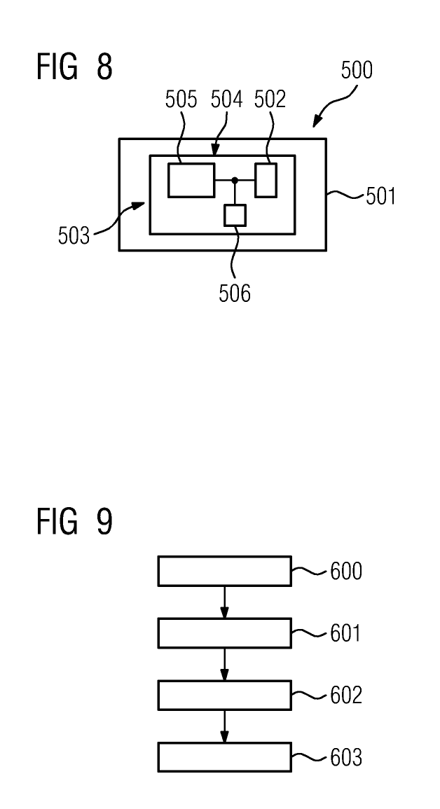

FIGS. 7 and 8 show an alternative embodiment of the magnetic resonance apparatus 400 to FIG. 6, with the magnetic resonance apparatus 400 also having an alternatively embodied hearing protection apparatus 500 to FIG. 1 to FIG. 5. The description which follows is essentially limited to the differences from the exemplary embodiment in FIGS. 1 to 5, with reference being made to the description of the exemplary embodiment in FIGS. 1 to 5 in respect of identical components, features and functions. Essentially identical components, features and functions are in principle shown with identical reference characters.

The hearing protection apparatus 500 in FIG. 8 is only shown schematically and can in principle comprise ear coupling units 501 configured by earmuffs or ear coupling units 501, which have at least one hearing protection unit that can be introduced within the auditory canal. Therefore FIG. 8 only shows one ear coupling unit 501 schematically in respect of its functionality, it being possible for the second ear coupling unit to be configured in a similar manner thereto or to be provided just with the communication unit 502 and the sound protection unit 503.

The hearing protection unit 500 in FIG. 8 comprises a motion sensor unit 504 with a sensor element 505, which emits and/or reflects a signal. The motion sensor unit 504 also has a power storage unit 506 to supply power to the sensor element 505.

The magnetic resonance apparatus 400 in FIG. 7 likewise has a motion sensor unit 401, which comprises individual sensor elements 402, which are disposed on a housing unit 404 of the magnetic resonance apparatus 400 enclosing the patient accommodation region 307. The individual sensor elements 402 detect the signal reflected and/or emitted by the sensor element 505 of the hearing protection unit 500. Based on this signal a data evaluation unit 405 of the magnetic resonance apparatus 400 determines movement of the head 17 of the patient 15 and transmits this movement information to the control unit 304 of the magnetic resonance apparatus 400.

Alternatively it is also conceivable for the sensor elements 402 of the motion sensor unit 401 of the magnetic resonance apparatus 400 to emit a signal and for the sensor element 505 of the motion sensor unit 504 of the hearing protection apparatus 500 to detect this signal to determine a head movement of the patient 15.

The hearing protection apparatuses 10, 100, 200, 500 described in FIGS. 1 to 8 can also have further functions, such as for example a unit for suppressing noise signals, etc.

FIG. 9 shows an inventive method for detecting movement of a head 17 of the patient 15 for magnetic resonance measuring in particular. In a first method step 600 a sensor signal is detected by means of the motion sensor unit 19, 108, 209, 401, 504 from FIGS. 1 to 8. Detection of the sensor signals takes place at least to some degree by means of the sensor elements 23, 24, 106, 107, 207, 208, 505, which are disposed within the hearing protection unit 10, 100, 200, 500 and/or by means of the sensor elements 402 disposed within the magnetic resonance apparatus 400. In a further method step 601 movement information is then determined by means of the data evaluation unit 405 or the electronic control unit 25, 109, 210. In this process the data evaluation unit 405 or the electronic control unit 25, 109, 210 evaluates the sensor signals in respect of a movement of the head 17 of the patient 15.

In a further method step 602 the determined movement information is then transmitted by means of the data transmission unit 27, 110, 211, 315 to the control unit 304 of the magnetic resonance apparatus 300, 400. In a further method step 603 a control signal for magnetic resonance measuring is generated, the control signal being generated as a function of the movement information determined from the sensor signals. If the control unit 304 identifies and/or determines movement of the head 17 of the patient 15 based on the movement information, the control signal can comprise a correction step. For example the patient 15 can be asked by the outputting of optical and/or acoustic instructions to lie still, in particular without moving, on a patient support apparatus 308. Magnetic resonance measuring can also be terminated immediately and restarted, thereby advantageously allowing the measuring time for the patient 15 to be shortened and/or just individual partial measurements to be repeated.

While specific embodiments have been described in detail, those with ordinary skill in the art will appreciate that various modifications and alternative to those details could be developed in light of the overall teachings of the disclosure. For example, elements described in association with different embodiments may be combined. Accordingly, the particular arrangements disclosed are meant to be illustrative only and should not be construed as limiting the scope of the claims or disclosure, which are to be given the full breadth of the appended claims, and any and all equivalents thereof. It should be noted that the term “comprising” does not exclude other elements or steps and the use of articles “a” or “an” does not exclude a plurality.

1. A hearing protection apparatus, comprising:
a first ear coupling unit designed for positioning on a first ear of a user;
a second ear coupling unit designed for positioning on a second ear of the user, and
a sound protection unit for at least partial shielding of sound waves; and
a motion sensor unit.

2. The hearing protection apparatus as claimed in claim 1,
wherein the motion sensor unit comprises a sensor element that detects a translational movement and/or a rotational movement of the head of the user.

3. The hearing protection apparatus as claimed in claim 2,
wherein the motion sensor unit comprises an acceleration sensor element.

4. The hearing protection apparatus as claimed in claim 2,
wherein the motion sensor unit comprises at least one gyroscopic sensor element.

5. The hearing protection apparatus as claimed in claim 2,
wherein motion sensor unit comprises a plurality of sensor elements.

6. The hearing protection apparatus as claimed in claim 5,
wherein the plurality of sensor elements are disposed within the first ear coupling unit or disposed within the second ear coupling unit.

7. The hearing protection apparatus as claimed in claim 5,
wherein at least a first of the plurality of sensor elements is disposed within the first ear coupling unit and at least a second of the at least two sensor elements disposed within the second ear coupling unit.

8. The hearing protection apparatus as claimed in claim 1,
wherein the motion sensor unit comprises an electronic control unit.

9. The hearing protection apparatus as claimed in claim 1,
wherein the motion sensor unit includes a data transmission unit.

10. The hearing protection apparatus as claimed in claim 9,
wherein the data transmission unit includes an antenna element.

11. The hearing protection apparatus as claimed in claim 1,
wherein the motion sensor unit comprises a power storage unit to supply power to the sensor elements.

12. The hearing protection apparatus as claimed in claim 1, comprising:
a communication unit for transmitting communication signals to a user.

13. The hearing protection apparatus as claimed in claim 9, comprising:
a communication unit for transmitting communication signals to a user.

14. The hearing protection apparatus as claimed in claim 13,
wherein the communication unit includes the data transmission unit, which is configured at least to some degree as a single piece with the data transmission unit of the motion sensor unit.

15. The hearing protection apparatus as claimed in claim 1,
wherein at least one of the first ear coupling unit and the second ear coupling unit comprises an earplug.

16. The hearing protection apparatus as claimed in claim 1,
wherein at least on of the first ear coupling unit and the second ear coupling unit comprises an earmuff.

17. A magnetic resonance apparatus comprising:
a patient support apparatus; and
a hearing protection apparatus as claimed in claim 1.

18. The magnetic resonance apparatus as claimed in claim 17,
wherein the hearing protection apparatus comprises a data transmission designed for data transmission with the motion sensor unit of the hearing protection apparatus.

19. The magnetic resonance apparatus as claimed in claim 17
wherein the hearing protection apparatus comprises a motion sensor unit which detects a sensor signal, which is emitted and/or reflected by at least one sensor element of the hearing protection apparatus.

20. The magnetic resonance apparatus as claimed in claim 17, comprising:
a data evaluation unit designed to evaluate the sensor signals.

21. The magnetic resonance apparatus as claimed in claim 20, comprising:
a control unit designed to control magnetic resonance measuring and take into account the evaluated data from the data evaluation unit when controlling magnetic resonance measuring.

22. A method for detecting movement of a head of a patient for magnetic resonance measuring, comprising:
detecting a sensor signal via a motion sensor unit, which is disposed at least partially within an ear coupling unit of a hearing protection apparatus;
determining movement information by evaluating the sensor signal via a data evaluation unit;
transmitting the movement information to a control unit; and
generating a control signal for magnetic resonance measuring as a function of the movement information.
